(19) United States
(12) Patent Application Publication
(10) Pub. No.: US 2007/0123834 A1
(43) Pub. Date: May 31, 2007

(54) Flexible absorbent article

(75) Inventors: Debra J. McDowall, Neenah, WI (US); Rhonda K. Joch, Neenah, WI (US); David L. Zenker, Neenah, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/287,683

(22) Filed: Nov. 28, 2005

(51) Int. Cl. A61F 13/15

(52) U.S. Cl. 604/367; 604/385.28

(57) ABSTRACT

An absorbent article having an outercover, liner and absorbent core that are each stretchable or extensible. The article has a thickness of about 4 mm or less, and a bending stiffness of less than about 6 N-mm. The tension in the waist elastic, flap elastics and/or leg elastics may be modified to accommodate the addition of a more flexible absorbent core. The bending stiffness of the fasteners may be reduced to further accommodate the improved flexibility of the overall article.

### BACKGROUND OF THE INVENTION

This invention is directed to an absorbent article, more specifically, an absorbent article worn about the lower torso and having the characteristics of being relatively thin and flexible.

Absorbent composites are incorporated into a variety of absorbent articles, including personal care products, health/medical products, and household/industrial products, for example. Conventional absorbent composites containing pulp fluff and super absorbent materials are typically not stretchable and as a result, not as flexible as cloth underwear. Combining a non-extensile absorbent member with new stretchable outercovers and/or stretchable body side liners may not always result in the most flexible absorbent article possible.

The softness and flexibility of an article made from new stretchable outercovers and/or stretchable body side liners can be enhanced by replacing an absorbent core that does not exhibit significant flexibility with an absorbent core that exhibits relatively high flexibility. However, there are several problems with fit that arise as the absorbent article becomes increasingly more flexible. For example, commonly used hook fasteners may experience failure. In particular, the edges of the fastener may at least partially disengage away from the loop portion of the fastener. To further exacerbate problems, a partially disengaged fastener can catch on clothing and become fully disengaged. Another challenge is buckling in the crotch and buttocks region. A stiffer absorbent material holds its shape during wear, whereas a flexible absorbent can lead to buckling in not only the absorbent area, but even the non-absorbent area. Buckling decreases the effective surface area of the absorbent member, resulting in a higher possibility of leakage. Yet another challenge is slippage and/or gapping of the fastened diaper from about the wearer's waist. A diaper with a flexible absorbent member or core can exhibit very different fit as compared to a diaper with a less flexible absorbent member.

As such, there is a need or desire for absorbent articles that exhibit more flexibility and softness while performing as well as or better than diapers that are less flexible.

### SUMMARY OF THE INVENTION

One embodiment of the present invention is an absorbent article with a stretchable outercover, a stretchable bodyside liner, and a pair of leg elastics for forming a pair of cuffs. The tension force in each of the pair of leg elastics at 50% extension is less than about 170 grams. An absorbent core, positioned between the outercover and the bodyside liner, is partially attached to the outercover. The absorbent core comprises at least 50% superabsorbent. The absorbent article has a total product thickness of less than 4 mm and a cylindrical compression of less than 600 grams.

Another embodiment of the present invention is an absorbent article that has a stretchable outercover, a stretchable bodyside liner, and an absorbent core positioned between the outercover and the bodyside liner. The absorbent core is partially attached to the outercover, and comprises at least 50% superabsorbent. A flexible fastener selectively attaches directly to the outercover, the fastener is a hook material having a stiffness of between about 10 and 120 mg. The absorbent article has a total product thickness of less than 4 mm and a bending stiffness of less than about 6.2 N-mm.

Yet another embodiment of the present invention is an absorbent article with an elastic outercover, an extensible bodyside liner, and an absorbent core positioned between the outercover and the bodyside liner. The absorbent core is partially attached to the outercover and comprises at least 50% superabsorbent, and at least 3% meltblown polymer. The absorbent article has a capacity of at least 0.3 g/cm2, and a total capacity of at least 200 grams. Further, the absorbent article has a total product thickness of less than 4 mm and a cylindrical compression of less than 600 grams.

Various other features, objects, and advantages of the invention will be made apparent to those skilled in the art from the following detailed description including illustrative examples setting forth various embodiments of the invention.

### BRIEF DESCRIPTION OF THE DRAWING

FIG. 1. representatively illustrates a partially cut away, plane view of an exemplary absorbent article in a stretched and laid flat condition with the surface of the article that contacts the skin of the wearer facing the viewer.

### DETAILED DESCRIPTION

Reference will now be made in detail to one or more embodiments of the invention, examples of which are graphically illustrated in the drawing. Each and every example and embodiment are provided by way of explanation of the invention, and not meant as a limitation of the invention. For example, features illustrated or described as part of one embodiment may be utilized with another embodiment to yield still a further embodiment. It is intended that the present invention include these and other modifications and variations.

The present invention provides an absorbent article that is soft and flexible thereby making the absorbent article feel more like cloth underwear. Most, if not all, absorbent article components influence the softness and flexibility of the article as it relates to material stiffness. For example, the outercover, the absorbent core, the liner, the fastener system and/or the diaper construction may influence the overall product softness and flexibility. The softness and flexibility of the present invention is provided though material selection, article design and article construction.

The absorbent article of the present invention will be described in terms of a diaper adapted to be worn by infants about the lower torso. It is understood that the absorbent article of the present invention is equally applicable to other articles such as adult incontinent garments (excluding pads), training pants, feminine care garments (excluding pads), and the like.

Within the context of this specification, each term or phrase below will include the following meaning or meanings.

“Elastomeric” and “elastic” are used interchangeably to refer to a material or composite that is generally capable of recovering its shape after deformation when the deforming force is removed. Specifically, as used herein, elastic or elastomeric is meant to be that property of any material which, upon application of a biasing force, permits the material to be stretchable to a stretched biased length which is at least about 50 percent greater than its relaxed unbiased length, and that will cause the material to recover at least 40 percent of its elongation upon release of the stretching force. A hypothetical example which would satisfy this definition of an elastomeric material would be a one (1) inch sample of a material which is elongatable to at least 1.50 inches and which, upon being elongated to 1.50 inches and released, will recover to a length of less than 1.30 inches. Many elastic materials may be stretched by much more than 50 percent of their relaxed length, and many of these will recover to substantially their original relaxed length upon release of the stretching force.

“Joined,” in reference to two materials or elements, is intended to refer to the situation wherein the two materials or elements are directly joined to one another or where they are indirectly joined to one another or where they are indirectly joined to an intermediate element. Similarly, methods of joining two materials or elements include forming the elements or materials integrally, or attaching the elements together such as through the use of adhesive bonds, sonic bonds, thermal bonds, pinning, stitching, or a variety of other attachment techniques known in the art, as well as combinations thereof.

“Absorbent article” includes, but is not limited to, personal care absorbent garments, and health/medical absorbent garments worn about the lower torso of the user.

“Layer” when used in the singular can have the dual meaning of a single element or a plurality of elements “Liquid-impermeable” when used to describe a layer or laminate means that liquid such as water or bodily fluids will not pass through the layer or laminate under ordinary use conditions in a direction generally perpendicular to the plane of the layer or laminate at the point of liquid contact.

“Liquid-permeable” refers to a layer or laminate that is not liquid-impermeable.

“Meltblown fiber” refers to fibers formed by extruding a molten thermoplastic material through a plurality of fine, usually circular or rectangular, die capillaries as molten threads or filaments into converging high velocity gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, and are generally self bonding.

“Nonwoven” and “nonwoven web” refer to materials and webs of material having a structure of individual fibers or filaments which are interlaid, but not in an identifiable manner as in a knitted fabric. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible configurational isomers of the material. These configurations include, but are not limited to isotactic, syndiotactic and atactic symmetries.

“Spunbond fiber” refers to small diameter fibers which are formed by extruding molten thermoplastic material as filaments from a plurality of fine capillaries of a spinnerette having a circular or other configuration, with the diameter of the extruded filaments then being rapidly reduced. Spunbond fibers are generally continuous.

“Stretchable” means that a material can be stretched, without breaking, by at least 30% (to at least 130% of its initial (unstretched) length) in at least one direction, suitably by at least 50% (to at least 150% of its initial length), or by at least 100% (to at least 200% of its initial length). The term stretchable includes elastic materials that are both extensible and retractable, as well as materials that stretch but do not significantly retract. A hypothetical example which would satisfy this definition of a stretchable material would be a one (1) inch sample of a material which could be elongated by at least 30% to at least 1.30 inches.

“Superabsorbent” refers to a water-swellable, water-insoluble organic or inorganic material capable, under the most favorable conditions, of absorbing at least about 10 times its weight, or at least about 15 times its weight, or at least about 25 times its weight in an aqueous solution containing 0.9 weight percent sodium chloride. The superabsorbent materials can be natural, synthetic, and modified natural polymers and materials. In addition, the superabsorbent materials can be inorganic materials, such as silica gels, or organic compounds such as cross-linked polymers. The superabsorbent material may be biodegradable or non-biodegradable. The superabsorbent materials can include particles, fibers, tows, flakes, films, foams, and the like.

These terms may be defined with additional language in the remaining portions of the specification.

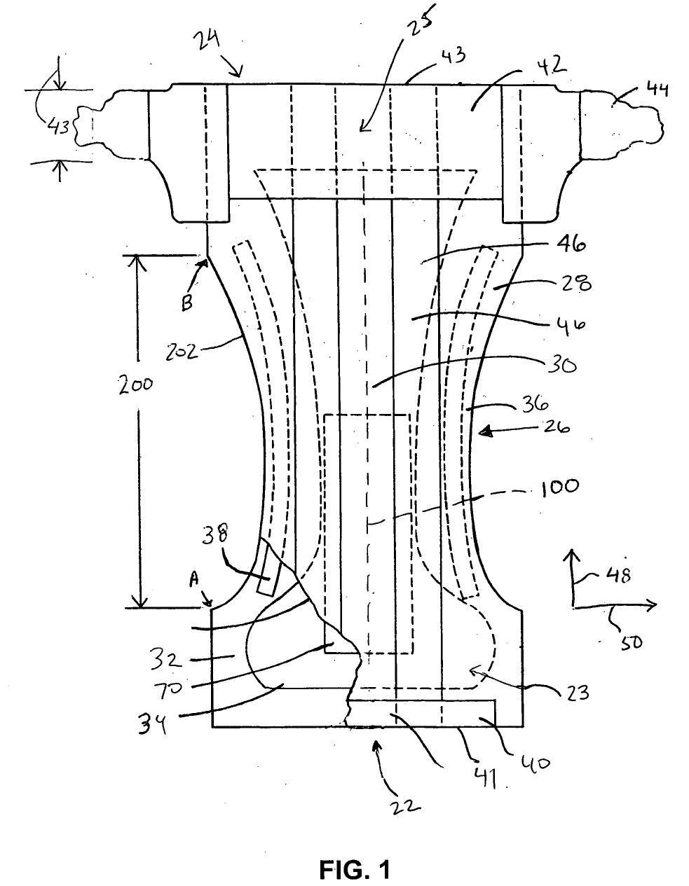

FIG. 1 representatively illustrates one exemplary embodiment of an absorbent article 20 of the present invention. The surface of the article which contacts the wearer is facing the viewer. The absorbent article 20 defines a front portion 22, a rear portion 24 and a crotch portion 26 connecting the front portion 22 and the rear portion 24. The front portion 22 defines a front waist region 23 and includes a front waist edge 41. The rear portion 24 defines a rear waist region 25 and includes a rear waist edge 43. The absorbent article 20 also defines a longitudinal direction 48 and a lateral direction 50. The absorbent article 20 includes a bodyside liner 30, an outercover 32 and an absorbent core 34 located between the bodyside liner 30 and the outercover 32.

As used herein, reference to a front portion refers to that part of the absorbent article which is generally located on the front of a wearer when in use. Reference to a front waist region 23 refers to that part of the front portion which is located generally near a waist opening that is defined when the absorbent article is in a fastened condition. Reference to the rear portion refers to the portion of the article generally located at the rear of the wearer when in use. Reference to a rear waist region 25 refers to that part of the rear portion which is located generally near the waist opening. Reference to the crotch portion 26 refers to that portion which is generally located between the legs of the wearer when in use.

The crotch portion 26 has opposite longitudinal side portions 28 which include a pair of elasticized, longitudinally-extending leg cuffs 36. The leg cuffs 36 are generally adapted to fit about the legs of a wearer in use and serve as a mechanical barrier to the lateral flow of body exudates. Each leg cuff 36 may be elasticized by one or more leg elastics 38. The absorbent article 20 may further include a front waist elastic 40 and/or a rear waist elastic 42.

The rear portion 24 of the absorbent article 20 may further include a fastener 44 which is intended to hold the absorbent article 20 about the waist of the wearer when in use. The absorbent article 20 may also include a pair of containment flaps 46 which extend longitudinally along the absorbent article 20 and are also adapted to provide a barrier to the flow of body exudates.

Leg and waist elastics may be sandwiched between two layers, or be located on an exposed surface of a layer. It should be recognized that individual components of the absorbent article 20, such as the leg and waist elastics, may be optional depending upon the intended use of the absorbent article 20.

A bodyside liner 30 may be suitably employed to help isolate the wearer's skin from fluids held in the absorbent core 34. The bodyside liner 30 of the absorbent article 20 suitably presents a bodyfacing surface which is intended to be worn adjacent the body of the wearer and is compliant, soft feeling and nonirritating to the wearer's skin. Further, the bodyside liner 30 may be less hydrophilic than the absorbent core 34, to present a relatively dry surface to the wearer, and may be sufficiently porous to be liquid permeable, permitting liquid to readily penetrate through its thickness. A suitable bodyside liner 30 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers.

The bodyside liner 30 may be stretchable or elastic. In one embodiment, the bodyside liner 30 may be a nonwoven, spunbond polypropylene fabric which has been neck-stretched up to approximately 40% of its original width. In other embodiments, the fabric can be neck-stretched less than 40% of its original width, for example, 25%. In other embodiments, the stretchable bodyside liner 30 can include elastic strands or netting, LYCRA® elastics, cast or blown elastic films, nonwoven elastic webs, meltblown or spunbond elastomeric fibrous webs, as well as combinations thereof. Examples of suitable elastomeric materials include KRATON® elastomers, HYTREL® elastomers, ESTANE® elastomeric polyurethanes (available from B.F. Goodrich and Company located in Cleveland, Ohio), PEBAX® elastomers, and elastomeric polyolefins such as VISTAMAXX (available from Exxon Mobil Corporation of Irving, Tex.), AFFINITY® (available from Dow Chemical of Midland, Mich.), and the like. The liner 30 may further be treated with a surfactant or otherwise processed to impart a desired level of wettability and hydrophilicity.

The outercover 32 of the absorbent article 20 may suitably be composed of a material which is either liquid permeable or liquid impermeable. In various embodiments, outercover 32 may be made from elastic or stretchable, fiber and film laminates. These laminates contribute to the overall product softness, flexibility and extensibility.

In one embodiment, the outercover 32 is made of an elastomeric material. Suitable elastomeric materials are stretchable in one or more directions. For example, in one embodiment, the outercover 32 may only stretch in the lateral direction 50. Elastomeric materials may include cast or blown films, foams, or meltblown fabrics composed of polyethylene, polypropylene, or polyolefin copolymers, as well as combinations thereof. The elastomeric materials may include PEBAX elastomer (available from AtoChem located in Philadelphia, Pa.), HYTREL elastomeric polyester (available from E.I. DuPont de Nemours of Wilmington, Del.), KRATON elastomer (available from Kraton Polymers of Houston, Tex.), or strands of LYCRA elastomer (available from E.I. DuPont de Nemours of Wilmington, Del.), or the like, as well as combinations thereof. The outercover 32 may include materials that have elastomeric properties through a mechanical process, printing process, heating process, or chemical treatment. For example, such materials may be apertured, creped, neck-stretched, heat activated, embossed, and micro-strained; and may be in the form of films, webs, and laminates.

In another embodiment, a biaxially stretchable outercover 32 is constructed from biaxially stretchable materials and/or biaxially elastic materials. One example of a suitable outercover material can include a 0.3 osy polypropylene spunbond that is necked 60% in the lateral direction and creped 60% in the longitudinal direction, laminated with 3 grams per square meter (gsm) adhesives H2525A, a styrene-isoprene-styrene based adhesive available from Bostik, Inc. in Wauwatosa, Wis., to 8 gsm PEBAX 2533 film with 20% TiO₂ concentrate. The outercover 32 can suitably be stretched, laterally and/or longitudinally, by at least 30% (to at least 130% of an initial (unstretched) width and/or length of the outercover 32). More suitably, the outercover 32 can be stretched laterally and/or longitudinally, by at least 50% (to at least 150% of the unstretched width or length of the outercover 32). Even more suitably, the outercover 32 can be stretched, laterally and/or longitudinally, by at least 100% (to at least 200% of the unstretched width or length of the outercover 32). Tension force in the outercover 32 at 50% extension is suitably between 50 and 1000 grams, or suitably between 100 and 600 grams, as measured on a 3 inch (7.62 cm) wide piece of the outercover material. Further examples of a biaxially stretchable outercover 32 are described in U.S. Pat. No. 5,883,028, issued to Morman et al., incorporated herein by reference to the extent that it is consistent (i.e., not in conflict) herewith. Other examples of materials having two-way stretchability and retractability are disclosed in U.S. Pat. No. 5,116,662, issued to Morman, and U.S. Pat. No. 5,114,781, issued to Morman, both of which are hereby incorporated herein by reference to the extent that each is consistent (i.e., not in conflict) herewith. These two patents describe composite elastic materials capable of stretching in at least two directions.

In yet an alternative embodiment, the outercover 32 is made of an extensible material. Extensible materials suitable for use as an outercover 32 can provide an elongation of at least 10%; alternatively, at least 20%; alternatively, at least 30%; or, alternatively, at least 40% when subjected to a tensile force of 30 gmf per inch (per 2.54 cm). Material suitable for use as an outercover 32 can also provide a substantially permanent deformation of at least 10%; alternatively, at least 15%; alternatively, at least 20%; alternatively, at least 25%; or, alternatively, at least 30% when subjected to a tensile force of 50 gmf per inch (per 2.54 cm) and then allowed to relax, after removal of the tensile force, for a period of 1 minute. It should be readily appreciated that the described removal of the applied force results in a substantially zero applied tensile stress/force.

The elastic or extensible outercovers 32 as described above may be used to engage hook-type fasteners thereby eliminating the need for an additional patch of material or fastener landing, e.g. loop, which can in some instances restrict stretch in the lateral direction 50. The advantages of not using an additional patch of material, commonly known as a “pub patch”, includes (a) fewer components to reduce cost and manufacturing time, (b) the absorbent article is more flexible as each additional layer added to the outercover may increase stiffness, and (c) improved fit in the lateral direction 50 due to the article more easily moving with the wearer. However, it is contemplated that a fastener landing could be used with such flexible materials as is known in the art.

The bodyside liner 30 and outercover 32 may be adhered to one another so as to form a chassis or pocket in which the composite absorbent core 34 is located. For example, they may be adhered directly to each other around the outer periphery of the absorbent article 20 by any means known to those skilled in the art such as adhesive bonds, sonic bonds or thermal bonds.

In one embodiment, the absorbent core 34 is partially attached to the outercover 32 so as to allow maximum flexibility of these components. In one example, the absorbent core 34 may be attached to outercover 32 at approximately the longitudinal axis of each respective component. More specifically, an attachment line 100 at which the outercover 32 is joined to the absorbent core 34 can be seen in FIG. 1. The attachment line 100 may be any type of bond such as a continuous or discontinuous line of adhesive, elastic adhesive, a continuous or discontinuous thermal bond, sonic bond, pressure bond, or the like. It is further contemplated that the absorbent core be attached at only three points along the attachment line 100; a bond at each end of the line 100, and at or near the mid-point of line 100. In another embodiment, the attachment line 100 is defined as two or more parallel lines of bonds, continuous or discontinuous, that are aligned substantially parallel to the longitudinal axis of the absorbent article 20. In another embodiment, the attachment is not along a straight line; for example, the attachment may be at each corner of the absorbent core 34, or along a zig-zag or s-shaped curve oriented along the longitudinal axis of the garment. Further, the attachment line 100 may be oriented laterally across the absorbent core. Many other possible attachment scenarios are contemplated, with the primary characteristic of maximizing the overall flexibility of absorbent article 20. However, the absorbent core 34 may be fully attached to the outercover 32 by a continuous adhesive layer or the like.

In conventional absorbent articles, the stiffness of a non-extensible absorbent core helps to prevent longitudinal buckling. Thus, by placing an extensible or stretchable absorbent core 34 in a diaper chassis, normally containing a non-extensible absorbent core, may contribute to crotch region 26 buckling in direction 48. In one embodiment of the present invention, the overall length of the absorbent article chassis is shortened to reduce bagginess or drooping in or near the crotch region 26. Shortening the chassis length (as defined by the outercover length) and the corresponding absorbent core 34 in longitudinal direction 48 to remove excess material will help to prevent bagginess or drooping. For example, a certain size chassis that is typically 476 mm in length may be shortened to about 434 mm; about an 8-10 percent reduction in length. Likewise, the corresponding absorbent core 34 may be shortened from 383 mm in length to about 353 mm, also about a 8-10 percent reduction in length.

Turning now to the leg cuffs 36 of the present invention, they can be suitably formed by portions of the outercover 32, and/or bodyside liner 30, which extend beyond the longitudinal sides of the composite absorbent core 34. Naturally, the leg cuffs 36 may also be formed from separate materials which are attached to the outercover 32 and/or bodyside liner 30.

As mentioned above, the leg cuffs 36 may include leg elastics 38. Leg elastics 38 are arranged to draw and hold the absorbent article 20 against the legs of the wearer. Materials suitable for use in forming leg elastics 38 are known to those skilled in the art. Exemplary of such materials are strands or ribbons of a polymeric, elastomeric material which are adhered to the absorbent article 20 in a stretched position, or which are attached to the absorbent article while the article is pleated, such that elastic constrictive forces are imparted to the absorbent article 20. In a particular aspect of the invention, the elastics may be composed of individual strands of LYCRA which are available from E. I. DuPont de Nemours Co., a business having offices in Wilmington, Del.

The leg elastics 38 may comprise a single strand of elastic material, or may comprise several parallel or non-parallel strands of elastic material and may be attached to the absorbent article 20 in any of several ways which are well known to those skilled in the art. For example, the elastics may be ultrasonically bonded, thermally bonded or adhesively bonded to the absorbent article 20.

The leg elastics 38 may be configured to accommodate a more flexible absorbent core. The leg elastics 38 are generally straight or optionally curved to more closely fit the contours of the legs and buttocks of the wearer and better contain bodily exudates. A relative reduction in tension along the leg elastic or leg cuff enhances fit by not pulling the article away from the waist of the wearer, and allows the absorbent core 34 to lie flatter or be less prone to buckling. In one example, reducing tension by about 25 percent results in an improved fit. Quantitatively, in the present invention, a leg elastic may have a tension force at 50% extension of less than about 170 grams, or less than about 165 grams, or even less than about 155 grams to enhance fit in a chassis as the absorbent article increases in flexibility as described herein. Possible ways of reducing tension of the leg cuff is to cut the leg elastics at various points along the length of the elastic, increase the length of the elastic, or use an elastic having lower tensile properties. However, the reduction of tension along the leg cuff is not limited to these methods.

In addition or in the alternative to having a leg elastic tension as described above, the leg cutout or contour of the leg cuff 36 may be changed. In general, decreasing the “convexity” of the leg cutout may improve fit, and moreover, may improve fit if the leg cut out substantially follows the sub-gluteal crease. Referring now to FIG. 1, convexity is described herein as the ratio of the leg opening perimeter (the sum of the linear distance 200 between points A and B and the leg length as measured along edge 202 between point A and point B) divided by the leg perimeter (the leg length as measured along edge 202 between point A and point B, multiplied by 2).
Convexity=leg opening perimeter/(2(leg perimeter))
Suitably, in the present invention, the convexity may be less than about 0.94, or less than about 0.92, or less than about 0.91, or less than about 0.88. As the leg cut-outs become more shallow, the convexity approaches a value of 1.0

Flaps 46 are optional, and if present, can exhibit a lower tension as compared to flaps used in absorbent articles having a less flexible absorbent core 34. For instance, in absorbent articles tested (Examples 7-9 of Table A), the flap tension level was reduced (as compared to Example 6 of Table A) to prevent buckling in the buttocks area. Thus, as with the leg elastics, proper flap tension can prevent buckling. For instance, each flap 46 may exhibit a tension of about 60 to 65 g, which is the load required to extend the flap as described in the test method herein. The total flap tensions (either single or double flaps) are suitably about 35 to about 60 grams, to about 35 to about 55 grams, to about 35 to 50 grams, or about 35 to 45 grams.

As mentioned above, waist elastics 40 and 42 may also be provided. Waist elastics 40 and 42 are also arranged to draw and hold the absorbent article 20 against the wearer. Materials suitable for use in forming waist elastics 40 and 42 are known to those skilled in the art. Exemplary of such materials are strands or ribbons of a polymeric, elastomeric material which are adhered to the absorbent article 20 in a stretched position, or which are attached to the absorbent article while the article is pleated, such that elastic constrictive forces are imparted to the absorbent article 20.

The waist elastics 40 and 42 may have any configuration which provides the desired performance. For example, waist elastics 40 and 42 may comprise a single strand of elastic material, neck bonded laminate (NBL), spunbond laminate (SBL) or may comprise several parallel or non-parallel strands of elastic material. The waist elastics 40 and 42 may be attached to the absorbent article 20 in any of several ways which are well known to those skilled in the art. For example, the elastics may be ultrasonically bonded, thermally bonded or adhesively bonded, or a combination thereof to the absorbent article 20. In addition, waist elastics 42 and 40 may have the surface area as shown in FIG. 1, or have a greater or lesser surface area as long as the desired tension is achieved.

As with the leg elastics, the waist may be configured to accommodate a more flexible absorbent core. Having a proper tension about the waist enhances fit by allowing the waist to more fully conform against the wearer's waist without gapping. As compared to the diaper in Example 6 of Table A, the tension may be relatively increased in both the front and back waist. The tension force in the front waist elastic at 50% extension may be greater than about 100 grams, or greater than about 120 grams, or even greater than about 135 grams. The tension force in the back waist elastic at 50% extension may be greater than about 180 grams, or greater than about 200 grams, or even greater than about 215 grams.

The fasteners 44 are typically applied to the corners of the rear portion 24 of the absorbent article 20 to provide a means for holding the article 20 on the wearer. Suitable fasteners 44 are well known to those skilled in the art and can include tape tab fasteners, hook and loop fasteners, mushroom and loop fasteners, snaps, pins, belts and the like, and combinations thereof. Typically, the fasteners 44 are configured to be refastenable. It should also be understood that it may be possible to dispense with the fastener 44 in an absorbent article having a given design configuration.

In some embodiments, the fastener 44 may be adapted to engage or otherwise join with the outercover 32, described previously. In other embodiments, a fastener landing material (not shown) is a piece of loop material (pub patch) located on the outercover 32 in the front waist region 23 and is adapted to engage hook-type fastener 44. In alternative embodiments, the fastener landing material may be a film adapted to engage with a tape tab fastener 44.

It is contemplated that fastener 44 can be of any shape, and/or may have a relatively small surface area for direct or indirect attachment to the outercover 32. In one embodiment as shown in FIG. 1, the shape of the fastener 44 may be such that it has the minimal length in direction 48 in which to adequately fasten an absorbent article under normal conditions of use. It has been determined by experimentation that excess fastener length in the longitudinal direction 48 can cause redness marking on the wearer's skin, and may cause the fastener 44 to become in advertently unfastened. One skilled in the art will realize that the actual length of fastener 44 is dependent on the size of the absorbent article 20 and the flexibility of the fastener material. However, it may be desirable to have a fastener 44 length that is less than about 40 mm, or less than about 30 mm.

The stiffness of the fastener may be measured according to the test method described herein. In one embodiment, the stiffness of the fastener is about 10 to about 120 milligrams, alternately about 10 to about 80 milligrams, and alternately about 10 to about 45 milligrams, as measured in at least the machine direction 48. In one embodiment, the fastener exhibits a stiffness that is equal to less that the front waist region 23 of article 20. The fastener 44 material may further be able to move with the article chassis during wear to prevent tab edges from lifting, while not marring the outercover 32 so that it does not bond well with the fastener 44 or appears fuzzy. One possible fastener 44 material is a hook material having the mechanical characteristics of VELCRO 85-1215. Other possible fasteners that may be used are disclosed in commonly owned patent application, US Patent Pub. 2003/0100878, incorporated herein to the extent that it does not contradict the present invention.

Referring again to FIG. 1, the absorbent core 34 is positioned between the bodyside liner 30 and the outercover 32 to form the absorbent article 20. The absorbent core 34 is generally conformable and capable of absorbing and retaining body exudates. It should be understood that, for the purpose of the present invention, the absorbent core 34 may comprise a single, integral piece of material or, alternatively, may comprise a plurality of individual separate pieces of material which are operably assembled together.

The absorbent core 34 may have any of a number of shapes and sizes. The absorbent core 34 may suitably be constructed from fibers, superabsorbent materials, extensible and/or stretchable materials. The absorbent core 34 may have an elastic, non-extensible or extensible core wrap material (not shown). An example of a fibrous material is a naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester and polyamide fibers; and synthetic fibers composed of a nonwettable thermoplastic polymer, such as polypropylene fibers, which have been hydrophilized by appropriate means known to those skilled in the art. The absorbent core 34 may also comprise selected blends of the various types of fibers mentioned above. In one embodiment, absorbent core 34 may contain at least 50% superabsorbent (described below), and may have a saturation capacity of greater than 0.3 g/cm2.

First Absorbent Core Example

In one embodiment, the absorbent core is a “stabilized” absorbent core as described in the following commonly owned patent and applications, incorporated herewith to the extent they are not contradictory: U.S. Pat. No. 6,362,389 to D. J. McDowall; U.S. Ser. No. 10/739,385 file Dec. 18, 2003; and U.S. Ser. No. 10/883,174 filed Jun. 30, 2004. In general, this particular absorbent core is stretchable, and includes the following materials and characteristics.

The elastomeric material of the polymer fibers may include an olefin elastomer or a non-olefin elastomer, as desired. In one embodiment, the elastomeric material of the polymer fibers may include KRATON blend G 2755 from Kraton Polymers; and various commercial grades of low crystallinity, lower molecular weight metallocene polyolefins available from ExxonMobil Chemical Company under the VISTAMAXX trade designation. The KRATON material is believed to be a blend of styrene ethylene-butylene styrene polymer, ethylene waxes and tackifying resins. The VISTAMAXX material is believed to be metallocene propylene ethylene co-polymer. Other examples are contemplated. The flexible polymer may include an operative amount of the elastomeric polymer fibers. The amount of flexible polymer in the absorbent composite 30 can be at least about 3 wt %, as determined with respect to the total weight of the composite, or about 15 wt %, or about 20 wt %. In another feature, the polymer fibers include an operative amount of a surfactant. The surfactant can be combined with the polymer fibers in any operative manner. Various techniques for combining the surfactant are conventional and well known to persons skilled in the art.

The absorbent structure 34 can also include a significant amount of base superabsorbent material, and the base superabsorbent material can desirably be in the form of particles or particulates. Superabsorbent materials suitable for use in the present invention are known to those skilled in the art, and may be in any operative form, such as particulate form. The amount of superabsorbent material in the absorbent composite 30 can be at least a minimum of about 50 wt %, as determined with respect to the total weight of the composite, or alternatively, be at least about 65 wt %, or alternatively be at least about 70 wt % or 75 wt % to provide improved benefits. In other aspects, the amount of superabsorbent material can be up to a maximum of about 95 wt %, or alternatively be up to about 90 wt %, or alternatively be up to about 85 wt % to provide desired effectiveness.

The elastomeric absorbent composite can further include a definite, discrete amount of hydrophilic fibers, such as cellulose or cellulosic fibers. The amount of hydrophilic fibers may be in an amount greater than 0 wt %, and can be at least about 5 wt % or about 7 wt % to provide desired benefits. In another aspect, the amount of cellulosic or other hydrophilic fibers can be up to a maximum of about 10 wt %, based upon the total weight of the elastomeric absorbent composite. The amount of hydrophilic fibers can alternatively be up to a maximum of about 20 wt %, and can optionally be up to about 40 wt %.

The cellulosic fibers may include, but are not limited to, chemical wood pulps such as sulfite and sulfate (sometimes called Kraft) pulps, as well as mechanical pulps such as ground wood, thermomechanical pulp and chemithermomechanical pulp. More particularly, the pulp fibers may include cotton, typical wood pulps, cellulose acetate, rayon, thermomechanical wood pulp, chemical wood pulp, debonded chemical wood pulp, milkweed floss, and combinations thereof. Pulps derived from both deciduous and coniferous trees can be used. Additionally, the cellulosic fibers may include such hydrophilic materials as natural plant fibers, cotton fibers, microcrystalline cellulose, microfibrillated cellulose, or any of these materials in combination with wood pulp fibers. Desired configurations of the absorbent composites of the invention can, for example, include a pulp fiber content which is in the range of about 0 to about 35 wt %.

Second Absorbent Core Example.

In one embodiment, the absorbent core is extensible, and is generally constructed from a combination of superabsorbent particles and adhesive fibers formed by deposition onto a substrate layer. An absorbent core of this type is described in commonly owned patent application, U.S. Ser. No. 10/880,996 filed Jun. 30, 2004, incorporated herewith to the extent that it is non-contradictory herewith. Generally, the absorbent core 34 may include about 40-99% by weight superabsorbent particles and about 1-60% by weight adhesive, suitably about 80-98% by weight superabsorbent particles and about 2-20% by weight adhesive, or suitably about 85-95% by weight superabsorbent particles and about 5-15% by weight adhesive. The absorbent core 34 may also include cellulose fibers and other ingredients.

The absorbent article 20 of the present invention may also contain a surge portion 70 to advantageously improve the overall fluid intake rate of the absorbent core 34. The surge portion 70 is typically less hydrophilic than the absorbent core 34 and is configured to collect and temporarily hold fluid surges. This configuration can also help prevent fluid exudates from pooling and collecting on portions of the absorbent core 34. Various woven and nonwoven materials can be used to construct the surge portion 70. For example, the surge portion 70 may be a layer of a spunbonded or meltblown web of polyolefin fibers or a bonded carded web of natural and/or synthetic fibers. The surge portion 70 may be a substantially hydrophobic material and, optionally, can be treated with a surfactant or otherwise to impart a desired level of wettability and hydrophilicity. The surge portion 70 may also include other wettable fiber materials such as cotton, rayon, wood pulp, inherently wettable synthetic polymers, hydrophilized or surface treated polymers and the like. The surge portion 70 may be of any desired shape and configuration.

Regardless of which absorbent core 34 configuration is used, suitable absorbent core 34 may have a capacity of greater than 0.3 g/cm2. The saturated retention capacity is a measure of the total absorbent capacity of the absorbent article.

The absorbent article of the present invention may have a thickness of less than 4 mm, preferably less than 3.5 mm, and more preferably less than 3 mm. The density of absorbent core 34 or other component of the absorbent article can be calculated from its basis weight and thickness. With respect to diapers, for example, the weight and thickness are measured on newly unpacked, and dry diapers at a restraining pressure of 0.2 psi (1.38 kPa).

Test Methods

Tensile Test For the purpose of further describing the present invention, the elastic tension of the containment flaps 46 of absorbent article constructed in accordance with the present invention is determined by the following test method, which is otherwise referred to herein as an elastic tension test. This test may be adapted to test the waist elastic and leg elastics. The description below is particular to the flaps 46. Each article to be tested is hung from a conventional lightbox or other suitable device in an unfolded, vertical orientation with one end (e.g., the back end) of the article up and the bodyside liner of the article facing laterally outward away from the lightbox. The lightbox includes a pair of fixed upper clamps spaced approximately 5.5 inches apart at their centerlines (e.g., spaced in accordance with the approximate spacing between the containment flaps 46 on the opposite sides of the article). The waistband 42 at the back end of the article is generally fully stretched (e.g., to eliminate gathers in or otherwise straighten the waistband) and the clamps are clamped to the waistband 42 without clamping any of the absorbent core therein. The operator then gently runs his or her fingers down the sides of the containment flaps to straighten and extend the article.

The waistband 40 at the front (i.e., lower) end of the article is then generally fully stretched (i.e., to eliminate gathers in or otherwise straighten the waistband) and a clamp weight is secured to the waistband 40 so that the article hangs freely from the upper clamps in a longitudinally elongated configuration. The clamp weight is approximately 1,000 grams total mass and includes a pair of clamps spaced approximately 5.5 inches apart at their centerlines (e.g., spaced approximately the same as the clamps attached to the back end of the article) for attaching the clamp weight to the front end of the article 20.

With the article 20 in this longitudinally elongated configuration, a pair of markings is made on each containment flap 46 generally where the elastics are attached to the flap. More particularly, the markings on each containment flap 46 are spaced longitudinally from each other a distance of about 178 mm and are equidistant from the longitudinal center of the flap. The clamp weight is then removed from the article and the article is removed from the lightbox. Each containment flap is cut from the article by first cutting the containment flap inward from the free (i.e., distal) edge of the flap to the secured edge of the flap at longitudinally spaced locations which are approximately 0.5 inches (12.85 mm) beyond each of the markings (e.g., toward the nearest end of the article) made on the flap, and then cutting lengthwise generally adjacent the bead of adhesive which secures the secured edge of the flap to the liner 30.

Each test specimen (e.g., the marked and cut portion of each containment flap 46) is then secured in a testing device by a generally fixed upper clip and a generally moveable lower clip, (available from Velcro USA, Inc located in Manchester, N.H.) both of which are constructed to inhibit the specimen against slipping or becoming damaged upon tensioning the specimen. More particularly, the specimen is first secured at one end by the upper clip, with the specimen marking near the one end being aligned with the leading edge (e.g., the lowermost edge) of the upper clip so that the specimen hangs freely from the upper clip in a generally relaxed (e.g., unstretched) condition. The weight of the specimen is tared and then the other end of the specimen is secured by the lower clip, with the marking near this end of the specimen being aligned with the leading edge (e.g., uppermost edge) of the lower clip. The lower clip is then moved longitudinally away from the upper clip until the specimen is elongated longitudinally to about 90% of the previously achieved longitudinally elongated configuration (e.g., to a configuration in which the spacing between the markings on the specimen is about 160 mm, which is 90% of 178 mm) of the article. The specimen is maintained in this condition for about thirty seconds. The elastic tension is then measured and recorded using a suitable force gauge. The containment flaps 46 of at least five like articles are tested using the identical procedure and the results are averaged to determine the elastic tension in the flaps 46.

Fastener Stiffness Test TAPPI Standard Test T 543 OM-94 is a suitable test procedure for measuring fastener stiffness. One suitable test apparatus is a Gurley Stiffness Tester Model 4171-D manufactured by Teledyne Gurley of Troy, N.Y.

Saturated Retention Capacity Test The saturated retention capacity is determined as follows. The absorbent garment to be tested, having a moisture content of less than about 7 weight percent, is weighed and then submerged in an excess quantity of room temperature (about 23 degrees Celsius) saline solution. The saline solution is an aqueous solution of about 0.9 percent sodium chloride by weight. A suitable product is S/P® certified blood saline, commercially available from Baxter Diagnostics in Deerfield, Ill. The garment is allowed to remain submerged for 20 minutes. After 20 minutes, the absorbent garment is removed from the saline solution and placed on a TEFLON® coated fiberglass screen having 0.25-inch openings (commercially available from Taconic Plastic Inc., Petersburg, N.Y.) which, in turn, is placed on a vacuum box and covered with a flexible rubber dam material. A vacuum of 3.5 kilopascals (0.5 pounds per square inch) is drawn in the vacuum box for a period of 5 minutes. The absorbent garment is weighed again. The amount of aqueous liquid retained by the absorbent garment is determined by subtracting the dry weight of the absorbent garment from the wet weight of the absorbent garment (after application of the vacuum) and is reported as the saturated retention capacity in grams of aqueous liquid retained.

Thickness Test The thickness of the absorbent article 20 can be measured in accordance with the following procedure. All measurements are made on newly unpacked absorbent articles within one hour from start of specimen preparation to completion of test. Unless otherwise stated, all tests are performed at a relative humidity of 50%+/−2% and a temperature of 73 degree F. Each absorbent article should be removed from its package and all elastics removed from the absorbent article to allow it to lie in a flat unwrinkled condition. The absorbent article must be handled carefully to avoid disturbing or changing the thickness.

A thickness tester, consisting of a digital indicator (Sony Model U30A, available from Sony Corporation, Tokyo, Japan) suitably attached to a comparator base (Starrett model 653G, available from L.S. Starrett Company, Athol, Mass.) is used. A 2.0 inch (5.08 cm) diameter circular platen made of brass is attached to the thickness indicator shaft which together with the shaft weight provides a pressure of 0.2 psi. The thickness indicator platen is first zeroed against the comparator base plate. The platen is then raised and the absorbent article is placed on the base plate, garment surface down. The absorbent article is positioned on the base plate so that when the platen is lowered it is in the region of the absorbent article for which the measurement is desired. The platen is then gently lowered onto the absorbent article. The absorbent article thickness is determined by reading the indicator 3.0 seconds after the platen comes in contact with the absorbent article.

Cylindrical Compression Test The method by which the Edge-wise Compression (EC) value can be determined is set forth below. A 2 inch by 12 inch (5.1 cm×30.5 cm) piece of absorbent material is cut with its longer dimension aligned with the longitudinal direction of the product or raw material web. The weight of the sample is determined. The thickness of the material is determined under a 0.2 psi (1.38 KPa) load. The material is formed into a cylinder having a height of 2 inches (5.1 cm), and with the two ends having 0-0.125 inch (0-3.18 mm) overlap, the material is stapled together with three staples. One staple is near the middle of the width of the product, the other two nearer each edge of the width of the material. The longest dimension of the staple is in the circumference of the formed cylinder to minimize the effect of the staples on the testing.

An INSTRON tester, or similar instrument is configured with a bottom platform, a platen larger than the circumference of the sample to be tested and parallel to the bottom platform, attached to a compression load cell placed in the inverted position. The specimen is placed on the platform, under the platen. The platen is brought into contact with the specimen and compresses the sample at a rate of 25 mm/min. The maximum force and the total energy obtained in compressing the sample to 50% of its width (1 inch) (2.54 cm) is recorded.

If the material buckles, it is typical for the maximum force to be reached before the sample is compressed to 50%. In a product where the length of the absorbent is less than 12 inches (30.5 cm), the EC value of the material can be determined in the following manner. A detailed discussion of the edge-wise compression strength has been given in The Handbook Of Physical And Mechanical Testing Of Paper And Parerboard, Richard E. Mark editor, Dekker 1983, (Vol. 1). Based on theoretical models governing buckling stresses, in the Edge-wise Compression configuration described, the buckling stress is proportional to E*t²/(H²) with the proportionality constant being a function of H²/(R*t) where E is the Elastic modulus, H is the height of the cylinder, R is the radius of the cylinder, and t is the thickness of the material. Expressing the stress in terms of force per basis weight, it can be shown that the parameter that needs to be maintained constant is H²/R. Therefore, for a sample that is smaller than 12 inches (30.5 cm), the largest possible circle should be constructed and its height (width of the sample being cut out) adjusted such that H²/R equals 2.1 inches (5.3 cm).
Bending Stiffness Test As used herein, the bending stiffness test is a measure of stiffness of a flat sample as it is deformed downward into a circular orifice beneath the sample. This test may be used to test absorbent and waist-band stiffness. For the test, the sample is modeled as an infinite plate with thickness t that resides on a flat surface where it is centered over an orifice with radius R. A central force applied to the sample directly over the center of the orifice deflects the sample down into the orifice by a distance w when loaded in the center by a Force F. For a linear elastic material the deflection can be predicted by:

$$w = \frac{3 F}{4 π E {t}^{3}} ( 1 - v ) ( 3 + v ) {R}^{2}$$

where E is the effective linear elastic modulus, v is the Poisson's ratio, R is the radius of the orifice, and t is the thickness of the sample, taken as the caliper in millimeters measured under a load of about 0.05 psi, applied by a 3-inch diameter Plexiglass platen, with the thickness measured with a Sony U60A Digital Indicator. Taking Poisson's ratio as 0.1 (the solution is not highly sensitive to this parameter, so the inaccuracy due to the assumed value is likely to be minor), the previous equation can be rewritten for w to estimate the effective modulus as a function of the flexibility test results:

$$E \approx \frac{2 {R}^{2}}{3 {t}^{3}} \frac{F}{w}$$

The test results are carried out using an MTS Alliance RT/1 testing machine (MTS Systems Corp., Eden Prairie, Minn.) with a 100 N load cell at a 100 Hertz data acquisition speed. As a 76.2 mm diameter sits centered over an orifice of 31.5 mm diameter on a support plate, a blunt probe of 6.35 mm diameter descends at a speed of 20.0 mm/min. The load cell monitors the applied force and the position of the probe tip relative to the plane of the support plate is also monitored. When the probe tip descends to 1 mm below the plane of the support plate, the test is terminated. The maximum slope in grams of force/mm over any 0.5 mm span during the test is recorded (this maximum slope generally occurs at the end of the stroke). The peak load is recorded, and E is estimated using the above equation.

The bending stiffness per unit width can then be calculated as:

$$S = \frac{E {t}^{3}}{12}$$

Experimental Data

The following Examples provide a comparison between diapers and pants that are currently or recently commercialized to diapers having optimized parameters of the present invention. Table A provides data for nine of the codes tested, which are described below.

TABLE A
Compression and Flexiblity Test Results for Sample Diapers and Pants

| | | | | Cylindrical | | | | |
|---|---|---|---|---|---|---|---|---|
| | | | | Comp. | | Flexibility | | |
| | | | | | Peak | | Bending | Bending |
| | | Basis | | Energy | Load | Peak | Modulus | Stiffness |
| Example | Thickness | Weight | Density | to 50% | to 50% | Load | (N- | (N- |
| (#) | (mm) | (g/m2) | (g/cm3) | (g * mm) | (g) | (g) | mm2) | mm) |
| 1 | 5.2 | 1113 | 0.18 | 59113 | 2725 | 1060 | 1.284 | 46.57 |
| 2 | 4.6 | 773 | 0.17 | 33337 | 1550 | 398 | 1.920 | 18.71 |
| 3 | 6.6 | 840 | 0.13 | 15930 | 1150 | 394 | 0.329 | 13.40 |
| 4 | 6.6 | 859 | 0.13 | 17989 | 1274 | 388 | 0.349 | 12.44 |
| 5 | 4.4 | 899 | 0.20 | 11217 | 789 | 225 | 0.241 | 8.01 |
| 6 | 4.7 | 1147 | 0.23 | 53594 | 2585 | 617 | 0.596 | 27.21 |
| 7 | 3.9 | 885 | 0.23 | 7048 | 403 | 188 | 0.495 | 6.18 |
| 8 | 3.6 | 916 | 0.26 | 6299 | 375 | 179 | 0.601 | 5.90 |
| 9 | 2.9 | 745 | 0.26 | 1159 | 68 | 19.6 | 0.289 | 0.76 |

Examples 1-2 are prior art absorbent articles sold by Kimberly-Clark Corporation r the trademark HUGGIES SUPREME (Lot PA419707F 16:33) and HUGGIES PULL-UPS (Lot # PA332412×13:45), respectively. These particular examples do not have a substantially elastic outercover or absorbent core.

Examples 3-4 are prior art absorbent articles sold by PROCTER AND GAMBLE under the trademark PAMPERS CRUISERS (Lot #4197UO176201:03) and PAMPERS EASY-UPS (Lot # 404?UG?????:32 [?=illegible] These particular examples do not have astic or extensible, outercover, liner, or absorbent core.

Example 5 is another product similar to the Example 1 HUGGIES SUPREME (Lot # B1408312A) as described above. In particular, the absorbent core in this embodiment is a mixture of about 65% superabsorbent and 35% fiberized wood pulp formed into an absorbent pad with a front basis weight of about 900 gsm, a back basis weight of about 450 gsm, and a density of about 0.24 g/cc.

Example 6 is a diaper-type article with an elastic outercover. In particular, the product is characterized as having the following specification:
Liner=21gsm polypropylene SB (spunbond) material necked 25%
Surge=76 gsm TABCW (through-air-bonded-carded-web)
Core wrap=13.6gsm wettable polypropylene SMS (spunbond-meltblown-spunbond) material
Absorbent core=a mixture of about 43% superabsorbent and 57% fiberized wood pulp formed into an absorbent pad with a front basis weight of about 970gsm, a back basis weight of about 550gsm, and a density of about 0.24 g/cc
The spacer layer is 13.6gsm SMS material
Outercover=CD elastic laminate, made from a 33gsm film made from a blend of 33% Septon 2004 elastomer and 67% Linear low density polyetylene/CaCO3 concentrate, adhesively laminated to a 0.6-0.65 osy necked SB facing

Example 7 is a product with a liner and outercover identical to that of Example 1 HUGGIES SUPREME (see above), but with another absorbent core embodiment of the present invention. In particular, the elastic absorbent core is characterized as having the following specification:
Absorbent core=a mixture of about 75% superabsorbent, 15% meltblown polymer, and 10% fiberized wood pulp formed into an absorbent pad with a basis weight of about 640gsm and a density of about 0.31 g/cc

Example 8 is an embodiment of an absorbent article in accordance with the present invention. This absorbent article is another diaper-type article, the article having an elastic outercover and an elastic absorbent core. In particular, the products are characterized as having the following specification:
Liner=21 gsm polypropylene SB material necked 25%
Surge=102 gsm TABCW
Core wrap=21gsm polypropylene spunbond necked 35%
Absorbent core=a mixture of about 75% superabsorbent, 15% meltblown polymer, and 10% fiberized wood pulp formed into an absorbent pad with a basis weight of about 640gsm and a density of about 0.31 g/cc
Spacer layer=13.6 gsm polypropylene SMS necked 25%
Outercover=CD elastic laminate as described in example 6

Example 9 is one embodiment of an absorbent article in accordance with the present invention. This absorbent article is another diaper-type article, the article having a biaxially elastic outercover and a elastic absorbent core. In particular, the products are characterized as having the following specification:
Liner=21 gsm polypropylene spunbond necked 25%
Absorbent core=a mixture of about 75% superabsorbent, 15% meltblown polymer, and 10% fiberized wood pulp formed into an absorbent pad with a basis weight of about 565gsm and a density of about 0.31 g/cc
Outercover=Biaxial elastic laminate, more specifically a film layer approximately 36gsm made from a blend of 40% Septon 2004 and 60% a linear low density polyethylene filled with calcium carbonate laminated with Bostik 9375 adhesive to an 0.8 osy elastic spunbond made from an 80/20 bicomponent sheath/core fibers with Affinity core/polyethylene sheath

When introducing elements of the present invention or the embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that the several objects of the invention are achieved and other advantageous results attained. As various changes could be made in the above constructions, products, and methods without departing from the scope of the invention, it is intended that all matter contained in the above description and shown in the accompanying drawings shall be interpreted as illustrative and not in a limiting sense.

1. An absorbent article comprising,
a stretchable outercover;
a stretchable bodyside liner;
a pair of leg elastics for forming a pair of cuffs, wherein the tension force in each of the pair of leg elastics at 50% extension is less than about 170 grams;
an absorbent core positioned between the outercover and the bodyside liner, the absorbent core being partially joined to the outercover, the absorbent core comprising at least 50% superabsorbent; and
wherein, the absorbent article has a total product thickness of less than 4 mm and a cylindrical compression of less than 600 grams.

2. The absorbent article of claim 1 wherein the absorbent core is partially attached to the outercover along an attachment line.

3. The absorbent article of claim 1 wherein the absorbent core has a capacity of at least 0.3 g/cm2.

4. The absorbent article of claim 3 wherein the absorbent core has a total capacity of at least 200 grams.

5. The absorbent article of claim 4 further comprising at least one flap adjacent to one of the pair of cuffs, the at least one flap having a tension force at 50% extension of less than about 35 to about 55 grams.

6. The absorbent article of claim 1 further comprising a leg cut out with a convexity ratio of less than about 0.94.

7. The absorbent of claim 1 further comprising a fastener attached to a waist region of the absorbent article, wherein the fastener is a hook material having a stiffness in the machine direction of less than about 120 mg.

8. The absorbent article of claim 1 further including a front waist elastic and a back waist elastic, wherein the tension force in the front waist elastic at 50% extension is greater than about 100 grams, and the and tension force in the back waist elastic at 50% extension is greater than about 180 grams.

9. The absorbent article of claim 1 wherein the tension force in one of the pair of leg elastics at 50% extension is less than about 155 grams.

10. An absorbent article comprising,
a stretchable outercover and a stretchable bodyside liner;
an absorbent core positioned between the outercover and the bodyside liner, the absorbent core being partially joined to the outercover, the absorbent core comprising at least 50% superabsorbent; and
a flexible fastener attached to a waist region of the absorbent article and that selectively attaches directly to the outercover, wherein the fastener is a hook material having a stiffness of between about 10 and 120 mg;
wherein, the absorbent article has a total product thickness of less than 4 mm and a bending stiffness of less than about 6.2 N-mm.

11. The absorbent of claim 10 wherein the absorbent article has a bending stiffness of less than 5 N-mm.

12. The absorbent article of claim 10 further comprising a leg cut out with a convexity ratio of less than about 0.94.

13. The absorbent of claim 12 wherein the absorbent article has a thickness of less than 3 mm.

14. The absorbent article of claim 10 wherein the absorbent core has a total capacity of at least 200 grams.

15. The absorbent article of claim 10 further including a pair of leg elastics wherein the tension force in one of the pair of leg elastics at 50% extension is less than about 170 grams.

16. The absorbent article of claim 10 further comprising a front waist elastic and a back waist elastic, wherein the tension force in the front waist elastic at 50% extension is greater than about 100 grams, and the tension force in the back waist elastic at 50% extension is greater than about 180 grams.

17. The absorbent of claim 16 further comprising a leg cut out with a convexity ratio of less than about 0.94.

18. The absorbent article of claim 17 further including a pair of flaps adjacent to the leg elastics.

19. The absorbent article of claim 16 wherein the tension force in the front waist elastic at 50% extension is greater than about 135 grams, and the and tension force in the back waist elastic at 50% extension is greater than about 215 grams.

20. An absorbent article comprising,
an elastic outercover;
an extensible bodyside liner;
an absorbent core positioned between the outercover and the bodyside liner, the absorbent core being partially attached to the outercover, the absorbent core
comprising:
at least 50% superabsorbent, and
at least 3% meltblown polymer, and
having a capacity of at least 0.3 g/cm2, and
a total capacity of at least 200 grams, and
wherein, the absorbent article has a total product thickness of less than 4 mm and a cylindrical compression of less than 600 grams.
